(19) United States
(12) Patent Application Publication
(10) Pub. No.: US 2024/0331823 A1
(43) Pub. Date: Oct. 3, 2024

(54) SYSTEMS AND METHODS FOR USING TREATMENT EFFECT MODELS FOR CARE MANAGEMENT INTERVENTIONS

(71) Applicant: Aetna Inc., Hartford, CT (US)

(72) Inventors: Eric Hamilton, Hartford, CT (US); Steven Felix, Hartford, CT (US); Yue Wang, Hartford, CT (US); Hyuna Yang, Hartford, CT (US); Youming Xu, Hartford, CT (US); Yiwei Jiang, Hartford, CT (US); Robin Foreman, Hartford, CT (US); Kirsten Wallace, Hartford, CT (US); Allison Freeman, Hartford, CT (US)

(21) Appl. No.: 18/130,149

(22) Filed: Apr. 3, 2023

(51) Int. Cl. G16H 20/00; G16H 10/60; G16H 50/30

(52) U.S. Cl. CPC G16H 20/00; G16H 10/60; G16H 50/30

(57) ABSTRACT

A method is provided. The method comprises: receiving, by a computing platform and from a plurality of data sources, population data for a plurality of first individuals; standardizing, by the computing platform, the population data to determine training data for a care management heterogeneous treatment effect (HTE) model; training, by the computing platform, the care management HTE model using the training data; determining, by the computing platform, a plurality of second individuals for care management interventions based on using the trained care management HTE model; and providing, by the computing platform and for display on a care management computing device, information indicating the plurality of second individuals for the care management interventions.

### BACKGROUND

In some instances, an enterprise organization may seek to provide interventions to an individual (e.g., a member or a patient). For instance, the individual may suffer from one or more chronic medical conditions such as diabetes or atherosclerotic cardiovascular disease and/or may be at a higher risk for falling or going to the emergency room (ER). Certain interventions performed by the enterprise organization such as care management interventions may be able to assist the individual in managing their conditions or reducing their risk for certain situations. However, while some individuals may benefit from care management interventions, other individuals might not benefit as much from the care management interventions. In fact, in some instances, individuals with the same medical condition or other risk factors may react differently to care management interventions. For instance, certain traits may impact whether the individual would benefit from care management interventions. Accordingly, there remains a technical need to determine and identify individuals that benefit from care management interventions.

### SUMMARY

In some examples, the present application is capable of performing care management identification using one or more heterogeneous treatment effect (HTE) models. For instance, individuals may exhibit a plurality of clinical impact factors such as chronic medical conditions, polypharmacy, social determinants of health (SDOH) risk, fall risk, medical non-adherence, avoidable ER risk, and so on. Based on their clinical impact factors, individuals may benefit from care management interventions. However, while certain individuals may benefit from the care management interventions, other individuals might not benefit from the care management interventions at all, or the care management interventions may have significantly less benefit to these individuals. Accordingly, a computing platform may use an HTE model to determine whether to target individuals for care management interventions.

For example, the computing platform may train a care management HTE model using training data. For instance, the computing platform may receive population data for a first set of individuals, which may include the clinical impact factors described above, covariates such as demographics, geographical location, and/or other information, past population engagement and outcome information associated with the first set of individuals (e.g., whether the first set of individuals were previously engaged in care management interventions), and/or clinical or financial outcomes associated with the first set of individuals after care management engagement. The computing platform may standardize the population data and train the care management HTE model using the standardized population data. Afterwards, the computing platform may receive information associated with a second set of individuals (e.g., clinical impact factors for the second set of individuals). Using the trained care management HTE model and the received information, the computing platform may determine one or more individuals from the second set of individuals for the care management interventions (e.g., to target and/or enroll into the care management interventions). For instance, using the trained care management HTE model, the computing platform may determine that certain individuals from the second set of individuals may benefit more than other individuals in the population from enrolling into the care management interventions. Accordingly, the computing platform may identify these individuals and may inform another device (e.g., provide information to a care management computing device) of these individuals. This will be described in further detail below.

In one aspect, a method is provided. The method comprises: receiving, by a computing platform and from a plurality of data sources, population data for a plurality of first individuals; standardizing, by the computing platform, the population data to determine training data for a care management heterogeneous treatment effect (HTE) model, wherein the training data comprises a plurality of impact factor datasets for the plurality of first individuals; training, by the computing platform, the care management HTE model using the training data, wherein the care management HTE model comprises one or more care management machine learning-artificial intelligence (ML-AI) models; determining, by the computing platform, a plurality of second individuals for care management interventions based on using the trained care management HTE model; and providing, by the computing platform and for display on a care management computing device, information indicating the plurality of second individuals for the care management interventions.

Examples may include one of the following features, or any combination thereof. For instance, in some examples, standardizing the population data comprises: determining a plurality of covariates for the care management HTE model based on the population data; determining the plurality of impact factor datasets for the care management HTE model based on the population data; and determining past population engagement in the care management interventions for the plurality of first individuals, wherein training the care management HTE model is based on the plurality of covariates, the plurality of impact factor datasets, and the past population engagement.

In some instances, standardizing the population data further comprises: determining an HTE outcome dataset for the care management HTE model based on training the care management HTE model using the plurality of covariates, the plurality of impact factor datasets, and the past population engagement, wherein the HTE outcome dataset comprises associated treatment effect model parameter values.

In some variations, determining the plurality of impact factor datasets comprises determining a plurality of impact factor metrics, wherein the plurality of impact factor metrics comprise fall risks, emergency room (ER) risks, medical adherence indicators, chronic condition counts, mental illness indicators, usage of durable medical equipment (DME), new onset of diseases, and/or drug safety indicators.

In some examples, training the care management HTE model using the training data comprises: using the plurality of impact factor datasets and the plurality of covariates to train the one or more care management ML-AI models, wherein the plurality of impact factor datasets and the plurality of covariates are features for the one or more care management ML-AI models.

In some instances, standardizing the population data further comprises: determining past population outcomes for the care management interventions for the plurality of first individuals, wherein the past population outcomes indicate post-engagement clinical and/or financial healthcare outcomes for the plurality of first individuals after undergoing the care management interventions, and wherein using the plurality of impact factor datasets and the plurality of covariates to train the one or more care management ML-AI models further comprises using the plurality of impact factor datasets, the plurality of covariates, the past population engagement, and the past population outcomes to train the one or more care management ML-AI models.

In some variations, determining the plurality of second individuals for the care management interventions based on using the trained care management HTE model comprises: determining a plurality of new impact factor and a plurality of new covariate datasets associated with the plurality of second individuals; inputting the plurality of new impact factor and the plurality of new covariate datasets into the one or more care management ML-AI models to determine output information for the plurality of second individuals; and determining the plurality of second individuals for the care management interventions based on the output information.

In some instances, determining the plurality of second individuals for the care management interventions comprises: combining the output information with additional metrics to generate combined strategic stratification metrics associated with the plurality of second individuals; and determining the plurality of second individuals for enrolling into the care management interventions based on comparing the combined strategic stratification metrics with one or more strategic stratification threshold values

In some examples, the plurality of first individuals and the plurality of second individuals are enrolled into MEDICARE.

In some variations, the plurality of first individuals and the plurality of second individuals are enrolled into MEDICAID.

In some instances, the plurality of first individuals and the plurality of second individuals are enrolled into a commercial plan

In another aspect, an enterprise computing platform is provided. The enterprise computing platform comprises: one or more processors; and a non-transitory computer-readable medium having processor-executable instructions stored thereon, wherein the processor-executable instructions, when executed by the one or more processors, facilitate: receiving, from a plurality of data sources, population data for a plurality of first individuals; standardizing the population data to determine training data for a care management heterogeneous treatment effect (HTE) model, wherein the training data comprises a plurality of impact factor datasets for the plurality of first individuals; training the care management HTE model using the training data, wherein the care management HTE model comprises one or more care management machine learning-artificial intelligence (ML-AI) models; determining a plurality of second individuals for care management interventions based on using the trained care management HTE model; and providing, for display on a care management computing device, information indicating the plurality of second individuals for the care management interventions.

Examples may include one of the following features, or any combination thereof. For instance, in some variations, standardizing the population data comprises: determining a plurality of covariates for the care management HTE model based on the population data; determining the plurality of impact factor datasets for the care management HTE model based on the population data; and determining past population engagement in the care management interventions for the plurality of first individuals, wherein training the care management HTE model is based on the plurality of covariates, the plurality of impact factor datasets, and the past population engagement.

In some instances, standardizing the population data further comprises: determining an HTE outcome dataset for the care management HTE model based on training the care management HTE model using the plurality of covariates, the plurality of impact factor datasets, and the past population engagement, wherein the HTE outcome dataset comprises associated treatment effect model parameter values.

In some examples, determining the plurality of impact factor datasets comprises determining a plurality of impact factor metrics, wherein the plurality of impact factor metrics comprise fall risks, emergency room (ER) risks, medical adherence indicators, chronic condition counts, mental illness indicators, usage of durable medical equipment (DME), new onset of diseases, and/or drug safety indicators.

In some variations, training the care management HTE model using the training data comprises: using the plurality of impact factor datasets and the plurality of covariates to train the one or more care management ML-AI models, wherein the plurality of impact factor datasets and the plurality of covariates are features for the one or more care management ML-AI models.

In some instances, standardizing the population data further comprises: determining past population outcomes for the care management interventions for the plurality of first individuals, wherein the past population outcomes indicate post-engagement clinical and/or financial healthcare outcomes for the plurality of first individuals after undergoing the care management interventions, and wherein using the plurality of impact factor datasets and the plurality of covariates to train the one or more care management ML-AI models further comprises using the plurality of impact factor datasets, the plurality of covariates, the past population engagement, and the past population outcomes to train the one or more care management ML-AI models.

In some examples, determining the plurality of second individuals for the care management interventions based on using the trained care management HTE model comprises: determining a plurality of new impact factor and a plurality of new covariate datasets associated with the plurality of second individuals; inputting the plurality of new impact factor and the plurality of new covariate datasets into the one or more care management ML-AI models to determine output information for the plurality of second individuals; and determining the plurality of second individuals for the care management interventions based on the output information.

In some variations, determining the plurality of second individuals for the care management interventions comprises: combining the output information with additional metrics to generate combined strategic stratification metrics associated with the plurality of second individuals; and determining the plurality of second individuals for enrolling into the care management interventions based on comparing the combined strategic stratification metrics with one or more strategic stratification threshold values.

In another aspect, a non-transitory computer-readable medium having processor-executable instructions stored thereon is provided. The processor-executable instructions, when executed, facilitate: receiving, from a plurality of data sources, population data for a plurality of first individuals; standardizing the population data to determine training data for a care management heterogeneous treatment effect (HTE) model, wherein the training data comprises a plurality of impact factor datasets for the plurality of first individuals; training the care management HTE model using the training data, wherein the care management HTE model comprises one or more care management machine learning-artificial intelligence (ML-AI) models; determining a plurality of second individuals for care management interventions based on using the trained care management HTE model; and providing, for display on a care management computing device, information indicating the plurality of second individuals for the care management interventions.

All examples and features mentioned above may be combined in any technically possible way.

### BRIEF DESCRIPTION OF THE DRAWINGS

The subject technology will be described in even greater detail below based on the exemplary figures, but is not limited to the examples. All features described and/or illustrated herein can be used alone or combined in different combinations. The features and advantages of various examples will become apparent by reading the following detailed description with reference to the attached drawings which illustrate the following:

FIG. 1 is a simplified block diagram depicting an exemplary computing environment in accordance with one or more examples of the present application.

FIG. 2 is a simplified block diagram of one or more devices or systems within the exemplary environment of FIG. 1.

FIG. 3 is an exemplary process for using the care management HTE model for care management interventions in accordance with one or more examples of the present application.

FIG. 4 is a simplified block diagram for training the care management HTE model in accordance with one or more examples of the present application.

FIG. 5 is another exemplary process for using the care management HTE model for care management interventions in accordance with one or more examples of the present application.

### DETAILED DESCRIPTION

Examples of the presented application will now be described more fully hereinafter with reference to the accompanying FIGs., in which some, but not all, examples of the application are shown. Indeed, the application may be exemplified in different forms and should not be construed as limited to the examples set forth herein; rather, these examples are provided so that the application will satisfy applicable legal requirements. Where possible, any terms expressed in the singular form herein are meant to also include the plural form and vice versa, unless explicitly stated otherwise. Also, as used herein, the term “a” and/or “an” shall mean “one or more” even though the phrase “one or more” is also used herein. Furthermore, when it is said herein that something is “based on” something else, it may be based on one or more other things as well. In other words, unless expressly indicated otherwise, as used herein “based on” means “based at least in part on” or “based at least partially on”.

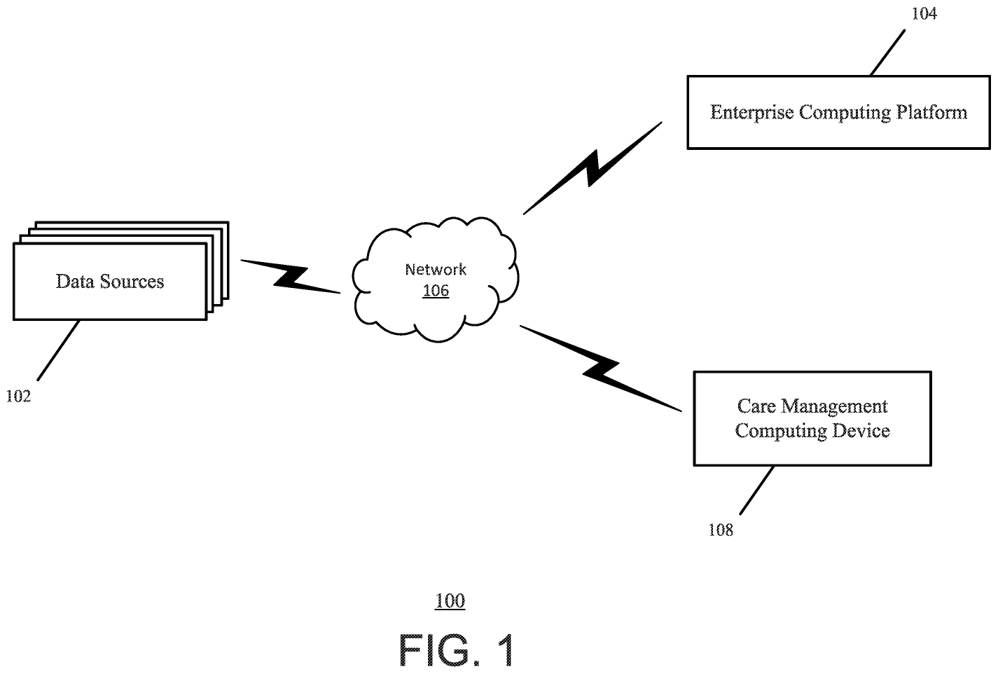

Systems, methods, and computer program products are herein disclosed that use one or more care management HTE models to identify individuals for care management interventions. FIG. 1 is a simplified block diagram depicting an exemplary environment in accordance with an example of the present application. The environment 100 includes a plurality of data sources 102, an enterprise computing platform 104, and a care management computing device 108. Although the entities within environment 100 may be described below and/or depicted in the FIGs. as being singular entities, it will be appreciated that the entities and functionalities discussed herein may be implemented by and/or include one or more entities. For instance, the enterprise computing platform 104 may include separate computing entities located in separate geographic locations that use the network 106 to communicate between each other, as well as other devices or entities within environment 100.

The entities within the environment 100 such as the data sources 102, the enterprise computing platform 104, and the care management computing device 108 may be in communication with other systems or facilities within the environment 100 via the network 106. The network 106 may be a global area network (GAN) such as the Internet, a wide area network (WAN), a local area network (LAN), or any other type of network or combination of networks. The network 106 may provide a wireline, wireless, or a combination of wireline and wireless communication between the entities within the environment 100.

Each of the data sources 102 is and/or includes one or more computing devices, platforms, and/or systems that are configured to provide information (e.g., data files or other types of information) to the enterprise computing platform 104. For example, the data sources 102 are and/or include one or more computing devices, computing platforms, systems, servers, desktops, laptops, tablets, mobile devices (e.g., smartphone device, or other mobile device), or any other type of computing device that generally comprises one or more communication components, one or more processing components, and one or more memory components.

The data sources 102 are capable of performing tasks, functions, and/or other actions associated with an enterprise organization. The data sources 102 may receive, generate, and/or otherwise obtain information associated with a plurality of individuals (e.g., members). Furthermore, the data sources 102 may store the information associated with the individuals. For instance, the data sources 102 may be and/or include an enterprise data warehouse (EDW), a production warehouse (PROD), a clinical platform data warehouse, a health care business warehouse (HCB), an external data warehouse, and/or one or more computing platforms (e.g., cloud computing platforms). The EDW may be a data warehouse that stores data for a plurality of individuals (e.g., members associated with the enterprise organization). For instance, the EDW may store identifiers for the members/individuals, control features, and/or member demographics (e.g., age, gender, and so on). The PROD may store risk scores for the plurality of individuals (e.g., risk scores associated with commercial members and/or Medicare members). The HCB may store records, procedures as well as other important information associated with the individuals (e.g., procedure records, lab records, and/or prescription records for the individuals). The clinical platform data warehouse may be a data warehouse that stores care management intervention activities and/or outcomes. The external data warehouse may store social determinant of health (SDOH) information for the individuals/members. The data sources 102 listed above are merely exemplary and the data sources 102 may include additional and/or alternative data sources 102 (e.g., one or more cloud computing platforms) that provide additional and/or alternative information to the enterprise computing platform 104.

In some variations, the data sources 102 may be implemented as engines, software functions, and/or applications. In other words, the functionalities of the data sources 102 may be implemented as software instructions stored in storage (e.g., memory) and executed by one or more processors.

The enterprise computing platform 104 is a computing platform that is associated with the enterprise organization. The enterprise organization may be any type of corporation, company, organization, and/or other institution. In some instances, the enterprise organization may own, operate, and/or be otherwise associated with a healthcare enterprise organization and/or an insurance institution. For instance, the enterprise organization may be associated with a provider or entity that provides care management interventions for individuals (e.g., patients and/or members). Care management interventions may include any type of clinical or wellness function, process, and/or program that is associated with care management (e.g., a patient-centered approach designed to assist patients and/or their support systems in managing medical conditions more effectively). For instance, care management interventions may include an operator, employee, other personnel, or automated method associated with the enterprise organization contacting the individuals (e.g., calling, e-mailing, texting, online/digital clinical or wellness programs, and/or otherwise contacting the individual) to enroll the individuals into care management. Additionally, and/or alternatively, care management interventions may include offering for a nurse, health coach, social worker, other healthcare practitioner, or other personnel to assist the individuals in managing their conditions such as by offering to review medications with the individuals, educate them on their medications, disease, or treatment plan, coordinate healthcare or social support services, provide education of their condition, and/or other types of actions or steps to manage the individual's symptoms, conditions, and/or risks. Additionally, and/or alternatively, care management interventions may include targeting, identifying, and/or enrolling individuals into care management. For instance, as will be explained below, the enterprise computing platform 104 may use an HTE model and/or one or more ML-AI models to determine individuals for care management interventions (e.g., individuals to be targeted for care management interventions, to be enrolled in care management interventions, and/or automatically enrolling individuals into care management interventions). Additionally, and/or alternatively, care management interventions may include actual care management and/or actual performance of care management programs (e.g., actual assistance provided to patients and/or their support systems to manage their medical conditions more effectively).

For example, individuals may have certain medical conditions and/or risk factors such as a risk of falling or diabetes. For instance, individuals that are enrolled into MEDICARE may be suffering from one or more medical conditions, including chronic conditions. These individuals may benefit from being enrolled into care management interventions (e.g., care management programs), which may allow a nurse to review medications with the individuals and/or allow a nurse to provide education of the individuals' medical condition(s) to the individuals. However, currently, there are a number of medical staffing shortages, which are due to many factors. Therefore, while it could be beneficial enroll each individual into care management interventions, it might not be practical to do so given the medical staffing shortages. In addition, certain individuals might not want to be enrolled into care management interventions in the first place. As such, as will be described in further detail below, the enterprise computing platform 104 may train a care management HTE model that includes one or more care management machine learning-artificial intelligence (ML-AI) models to determine (e.g., identify) individuals for care management interventions (e.g., identify individuals to enroll or target for care management interventions). Then, the enterprise computing platform 104 may provide information indicating these individuals to another device such as the care management computing device 108. The care management computing device 108 may perform aspects of enrolling and/or implementing the care management interventions for the identified individuals.

The enterprise computing platform 104 includes one or more computing devices, computing platforms, systems, servers, and/or other apparatuses capable of performing tasks, functions, and/or other actions for the enterprise organization. For instance, the enterprise computing platform 104 may include a training system that is configured to train the one or more care management HTE models, which may include one or more care management ML-AI models. The enterprise computing platform 104 may further include a determination system that is configured to use the trained care management HTE models to determine or identify the individuals to be enrolled into the care management interventions. In some variations, the enterprise computing platform 104 may be implemented as engines, software functions, and/or applications. In other words, the functionalities of the enterprise computing platform 104 may be implemented as software instructions stored in storage (e.g., memory) and executed by one or more processors.

The care management computing device 108 may be and/or include, but is not limited to, a desktop, laptop, tablet, mobile device (e.g., smartphone device, or other mobile device), smart watch, an internet of things (IoT) device, or any other type of computing device that generally comprises one or more communication components, one or more processing components, and one or more memory components. The care management computing device 108 may be able to execute software applications managed by, in communication with, and/or otherwise associated with the enterprise organization. Additionally, and/or alternatively, the care management computing device 108 may be configured to perform other functions. For instance, the care management computing device 108 may perform care management interventions and/or actual care management for one or more individuals. For instance, the care manage computing device 108 may display information indicating the one or more individuals. Based on the displayed information, an operator may contact the individuals to enroll them into care management interventions.

It will be appreciated that the exemplary environment depicted in FIG. 1 is merely an example, and that the principles discussed herein may also be applicable to other situations—for example, including other types of institutions, organizations, devices, systems, and network configurations. As will be described herein, the environment 100 may be used by health care enterprise organizations. However, in other instances, the environment 100 may be used by other types of enterprise organizations such as financial institutions or insurance institutions.

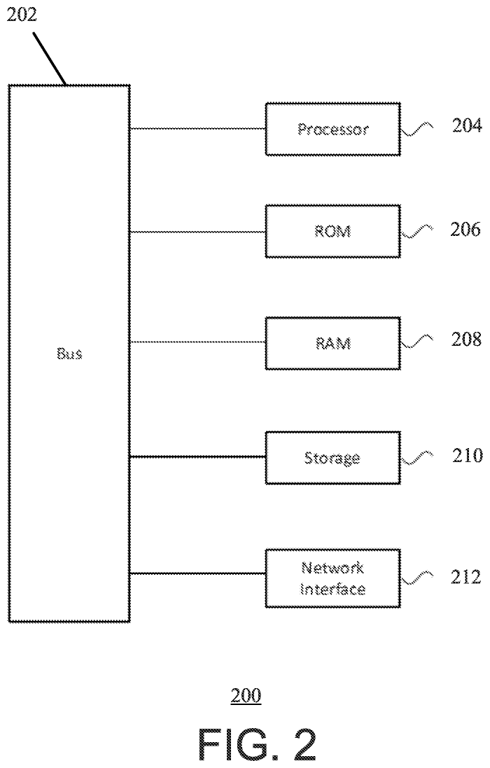

FIG. 2 is a block diagram of an exemplary system and/or device 200 within the environment 100. The device/system 200 includes a processor 204, such as a central processing unit (CPU), controller, and/or logic, that executes computer executable instructions for performing the functions, processes, and/or methods described herein. In some examples, the computer executable instructions are locally stored and accessed from a non-transitory computer readable medium, such as storage 210, which may be a hard drive or flash drive. Read Only Memory (ROM) 206 includes computer executable instructions for initializing the processor 204, while the random-access memory (RAM) 208 is the main memory for loading and processing instructions executed by the processor 204. The network interface 212 may connect to a wired network or cellular network and to a local area network or wide area network, such as the network 106. The device/system 200 may also include a bus 202 that connects the processor 204, ROM 206, RAM 208, storage 210, and/or the network interface 212. The components within the device/system 200 may use the bus 202 to communicate with each other. The components within the device/system 200 are merely exemplary and might not be inclusive of every component within the device/system 200.

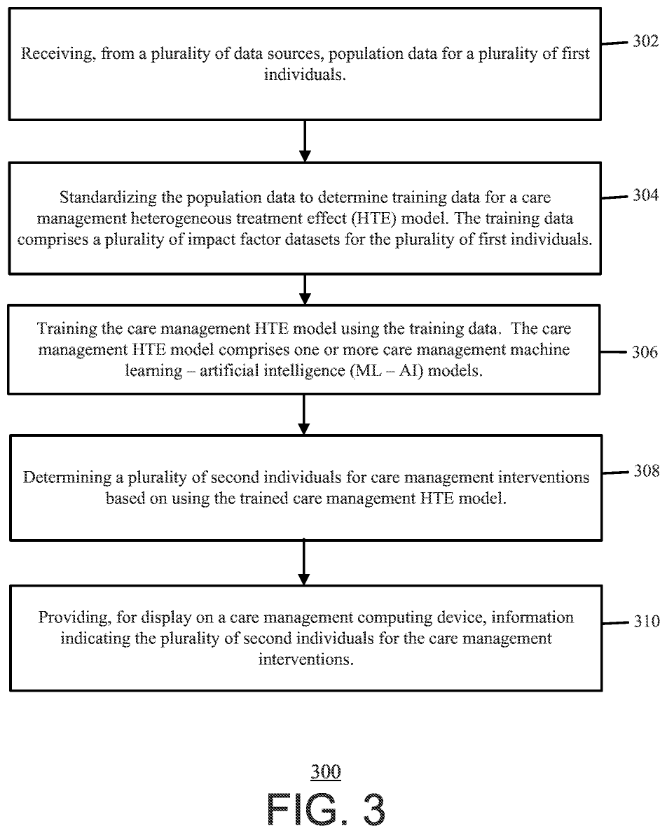

FIG. 3 is an exemplary process for using the care management HTE model for care management interventions in accordance with one or more examples of the present application. The process 300 may be performed by the enterprise computing platform 104 shown in FIG. 1. However, it will be recognized that any of the following blocks may be performed in any suitable order and that the process 300 may be performed in any suitable environment. The descriptions, illustrations, and processes of FIG. 3 are merely exemplary and the process 300 may use other descriptions, illustrations, and processes.

At block 302, the enterprise computing platform 104 receives, from a plurality of data sources (e.g., data sources 102), population data for a plurality of first individuals. The first individuals may include a set of individuals that have previously engaged in care management interventions (e.g., actual engagement in care management programs) and another set of individuals that have not engaged in care management interventions. The other set of individuals may be a control group that qualified for care management interventions, but were ultimately not enrolled into care management interventions. For example, the enterprise computing platform 104 may be associated with an enterprise organization that provides services (e.g., insurance services) to a plurality to individuals (e.g., members). The enterprise computing platform 104 may store information (e.g., population data) associated with the plurality of individuals. The population data may be and/or include any type of information that is associated with the first individuals. For example, as will be explained below, the enterprise computing platform 104 may use the population data to determine covariate information, impact factor datasets, and/or past population engagement and outcome information for the first individuals.

At block 304, the enterprise computing platform 104 standardizes the population data to determine training data for a care management heterogeneous treatment effect (HTE) model. The training data comprises a plurality of impact factor datasets for the plurality of first individuals. For example, by standardizing the population data, the enterprise computing platform 104 may generate training data comprising covariate information, impact factor datasets, and/or past population engagement and outcome information for the first individuals.

The covariate information may indicate information such as personal information associated with the first individuals. For instance, the covariate information may indicate demographics (e.g., age, gender, and/or marital status) for the first individuals. Additionally, and/or alternatively, the covariate information may indicate pre-period (e.g., pre-engagement and/or pre-identification) allowed medical costs per member per month (PMPM) by medical cost category (MCC) for the first individuals. The covariate information may indicate pre-period medical utilization (e.g., inpatient (IP) admits/days, emergency room (ER) visits) for the first individuals. Additionally, and/or alternatively, the covariate information may indicate chronic conditions associated with the first individuals. Additionally, and/or alternatively, the covariate information may indicate geography associated with the first individuals such as a geographic location (e.g., address, county, city, state, and/or region) of where the first individuals reside. Additionally, and/or alternatively, the covariate information may indicate risk scores for the first individuals such as inpatient risk scores and/or financial risk scores. The inpatient risk scores may indicate a risk that the first individuals may be admitted into a medical facility (e.g., the individual is admitted into inpatient care). The financial risk scores may indicate health-care expenditures, and/or other healthcare related financial information associated with the first individuals. In some examples, the enterprise computing platform 104 may receive information associated with the first individuals and use one or more ML-AI models to determine the inpatient and/or financial risk scores associated with the first individuals. The covariate information described above is merely exemplary and may include additional and/or alternative covariate information.

The impact factor datasets may indicate features that are used to determine (e.g., estimate) the impact of enrolling individuals into care management interventions (e.g., actual care management). For instance, the impact factor datasets may include impact factor metrics. For example, the impact factor metrics may indicate avoidable ER risk and/or fall risk for the first individuals (e.g., risk of the individuals being admitted into the ER and/or falling). Additionally, and/or alternatively, the impact factor metrics may indicate medical treatment plan adherence or non-adherence indicators for the first individuals (e.g., the individuals adhered to or did not adhere to their medical treatment, prescription, advice from physicians, and/or other types of medical adherence). Additionally, and/or alternatively, the impact factor metrics may indicate chronic condition counts and/or information for the first individuals (e.g., whether the individuals suffer from chronic conditions, including major chronic conditions, high severity chronic conditions, mental illness, new onsets of chronic conditions, and/or other types of medical conditions). Additionally, and/or alternatively, the impact factor metrics may indicate SDoH indices for the first individuals (e.g., scaled individual-level SDoH metrics for the first individuals such as education level, household wealth, and housing stability). Additionally, and/or alternatively, the impact factor metrics may indicate polypharmacy for the first individuals (e.g., the individual is using multiple prescriptions to treat an ailment or condition). Additionally, and/or alternatively, the impact factor metrics may indicate the usage of durable medical equipment (DME) by the first individuals (e.g., usage of medical equipment to aid in a better quality of living for the first individuals). Additionally, and/or alternatively, the impact factor metrics may indicate expected and/or unexpected inpatient admissions for the first individuals (e.g., whether the medical conditions of the individual caused expected or unexpected inpatient admissions). The impact factor datasets and/or metrics described above are merely exemplary and may include additional and/or alternative impact factor datasets.

The past population engagement and outcome information may indicate previous engagement or involvement in care management interventions (e.g., actual care management). For example, a portion of the first individuals (e.g., a first subset) may have engaged in care management interventions. Another portion of the first individuals (e.g., a second subset) may have qualified for care management interventions, but did not engage in care management interventions. The past population engagement and outcome information may indicate the individuals from the plurality of first individuals that engaged in care management interventions and/or did not engage in care management interventions for a previous period (e.g., a previous time period). For instance, the past population engagement and outcome information may include indicators for the first individuals, and the indicators may indicate whether the first individuals engaged in care management interventions.

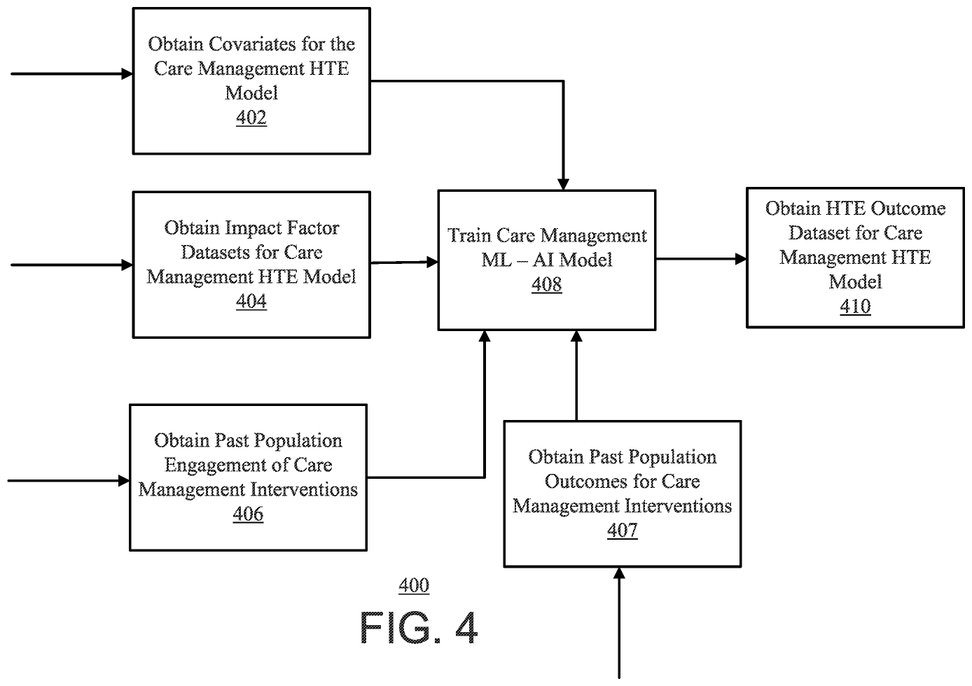

Additionally, and/or alternatively, the past population engagement and outcome information may indicate outcomes associated with the plurality of first individuals that engaged in care management interventions. For instance, the past population engagement and outcome information may indicate outcomes such as post-period utilization and medical costs for the first individuals that engaged in care management interventions. As will be explained below in FIG. 4, the outcomes from the past population engagement and outcome information may be used to train the HTE model.

In other words, in some examples, the enterprise computing platform 104 may determine the covariates, the impact factor datasets, and the past population engagement and outcome information from the population data for the first individuals. The covariates may be any type of variable associated with the first individuals that can influence an outcome of care management interventions, and thus may also influence the HTE model, but is not of direct interest to the HTE model. The impact factor datasets may be any type of variable associated with the first individuals that are of direct interest and can influence an outcome of the HTE model. The past population engagement and outcome information may be indicators indicating which individuals from the first individuals were enrolled into care management interventions. Additionally, and/or alternatively, the past population engagement and outcome information may indicate outcomes associated with the first individuals that engaged in care management interventions.

In some instances, the enterprise computing platform 104 may use one or more data structures (e.g., tables, arrays, and/or other types of data structures) to standardize the population data into the training data (e.g., the covariate information, the impact factor datasets, and/or the past population engagement and outcome information for the first individuals). For instance, the enterprise computing platform 104 may use a data structure that indicates features used to train the care management HTE models. The features may include and/or be associated with the covariate information, the impact factor datasets, and/or the past population engagement and outcome information for the first individuals. For example, the enterprise computing platform 104 may populate the data structure with the information from the population data. For instance, the population data may indicate demographics for the first individuals and/or chronic medical conditions for the first individuals. The enterprise computing platform 104 may standardize the population data and generate the training data based on filtering through the population data and populating entries from the data structure based on the population data. For instance, the enterprise computing platform 104 may populate the entries (e.g., features) for the demographics and/or chronic medical conditions for the first individuals based on the population data indicating the demographics and/or chronic medical conditions for the first individuals.

In some examples, the first individuals may be associated with a particular insurance and/or healthcare program such as, but not limited to, MEDICARE, MEDICAID, and/or commercial. For example, the first individuals may be enrolled into MEDICARE. For instance, the enterprise computing platform 104 may receive population data for individuals (e.g., members) that are enrolled into MEDICARE. Based on the population data, the enterprise computing platform 104 may standardize the population data to determine training data (e.g., training data for individuals enrolled into MEDICARE). The enterprise computing platform 104 may train the care management HTE model described below using the training data for individuals enrolled into MEDICARE. Therefore, in such examples, the care management HTE model may be a model for MEDICARE individuals.

In other examples, the first individuals may be enrolled into MEDICAID. For instance, the enterprise computing platform 104 may receive population data for individuals (e.g., members) that are enrolled into MEDICAID. Based on the population data, the enterprise computing platform 104 may standardize the population data to determine training data (e.g., training data for individuals enrolled into MEDICAID). The enterprise computing platform 104 may train the care management HTE model described below using the training data for individuals enrolled into MEDICAID. Therefore, in such examples, the care management HTE model may be a model for MEDICAID individuals.

In yet other examples, the first individuals may be enrolled into a commercial insurance plan. For instance, the enterprise computing platform 104 may receive population data for individuals (e.g., members) that are enrolled into the commercial insurance plan. Based on the population data, the enterprise computing platform 104 may standardize the population data to determine training data (e.g., training data for individuals enrolled into the commercial insurance plan). The enterprise computing platform 104 may train the care management HTE model described below using the training data. Therefore, in such examples, the care management HTE model may be a model for individuals enrolled into a commercial insurance plan.

In yet other examples, the population data for the first individuals may be associated with individuals enrolled into MEDICARE, MEDICAID, and/or a commercial insurance plan. In such examples, the enterprise computing platform 104 may train multiple care management HTE models. For example, the enterprise computing platform 104 may filter the population data to determine MEDICARE training data and MEDICAID training data. Using the training data, the enterprise computing platform 104 may train one or more first care management HTE models for MEDICARE and one or more second care management HTE models for MEDICAID. Additionally, and/or alternatively, the enterprise computing platform 104 may train a single care management HTE model for MEDICARE, MEDICAID, and/or the commercial insurance plan.

At block 306, the enterprise computing platform 104 trains the care management HTE model using the training data. The care management HTE model comprises one or more care management ML-AI models. For example, using the training data determined at block 304 (e.g., the covariate information, the impact factor datasets, and/or the past population engagement and outcome information for the first individuals), the enterprise computing platform 104 may train the care management HTE model. The care management HTE model may be any type of treatment effect model (e.g., a heterogeneous treatment effect model) that evaluates the effect of care management interventions (e.g., actual care management) on individuals. For example, using the care management HTE model, the enterprise computing platform 104 may determine the causal effect of care management interventions on the health and/or well-being of individuals.

The care management HTE model may include one or more care management ML-AI models. The care management ML-AI models may be any type of ML-AI model (e.g., unsupervised, supervised, and/or deep learning) that can be used to determine (e.g., identify) individuals to be enrolled into care management interventions. For instance, the care management ML-AI model may be an extreme Gradient Boosting (XGBoost) regression and/or classifier. Additionally, and/or alternatively, the care management ML-AI model may be a causal forest model and/or EconML.

In some examples, the enterprise computing platform 104 may produce (e.g., determine or generate) an HTE outcome dataset for the care management HTE model based on training the care management HTE model. The HTE outcome dataset may indicate the performance of the care management HTE model (e.g., a percentage of actual health outcome variation explained, differences between actual and predicted outcome values, and/or other indicators of model performance). The health outcomes may be a measure of health service utilization (e.g. hospital admission, emergency room visits), health care cost, or other outcomes or means to measure the healthiness of an individual after engaging in care management interventions. Additionally, and/or alternatively, the HTE outcome dataset may indicate best performing (e.g. most accurate) treatment effect model parameter values (e.g., HTE model hyperparameters that specify how the HTE model will operate when implemented) and model object parameters (e.g., parameters specific to input data elements such as impact factor and covariate weights, estimates, and/or other values) that transform the input dataset in a manner that produces the estimated treatment effect for individuals in the training dataset.

After training the care management HTE model and/or the care management ML-AI models, the enterprise computing platform 104 may store the care management HTE model and/or the care management ML-AI models in memory. Then, as described below, the enterprise computing platform 104 may retrieve and use the care management HTE model and/or the care management ML-AI models to identify new individuals for care management interventions.

At block 308, the enterprise computing platform 104 determines a plurality of second individuals for care management interventions (e.g., enrolling into actual care management and/or care management programs) based on using the trained care management HTE model. For example, the enterprise computing platform 104 may receive and/or obtain data associated with the second individuals. For instance, the second individuals may be part of a larger group of individuals (e.g., a plurality of third individuals), which may or might not include one or more individuals from the plurality of first individuals. The enterprise computing platform 104 may receive information from the plurality of third individuals and standardize the information. For instance, based on standardizing the received information, the enterprise computing platform 104 may determine impact factor datasets and/or covariate datasets (e.g., new impact factor and/or covariate datasets) for the plurality of third individuals. The determined impact factor datasets for the third individuals includes the same features as the impact factor datasets for the first individuals described above. For instance, the impact factor datasets for the third individuals may include information indicating SDoH indices, avoidable ER risk, fall risk, medical adherence indicators, chronic condition counts and/or information, and/or other types of impact factor datasets. For example, the enterprise computing platform 104 may filter the received information to determine the impact factor datasets for the third individuals, and/or populate a data structure based on the impact factor datasets.

Furthermore, the enterprise computing platform 104 may input information such as the determined impact factor datasets into the trained care management HTE model to determine output information. The output information may indicate the plurality of second individuals. For example, the enterprise computing platform 104 may determine impact factor datasets for the third individuals, and use the trained care management HTE model to identify certain individuals (e.g., the second individuals) from the third individuals. The second individuals may be identified as responding more favorably to care management interventions (e.g., actual care management). For instance, as mentioned previously, it may be difficult to offer care management interventions to every individual associated with the enterprise organization. As such, the enterprise computing platform 104 may determine certain individuals (e.g., the second individuals) from a group of individuals (e.g., the third individuals) that may respond more favorably to care management interventions. For instance, by enrolling the second individuals into care management interventions, this may result in fewer adverse medical events (e.g. unplanned hospital admissions or ER visits) and/or medical cost savings for the individuals and/or the enterprise organization. The enterprise computing platform 104 may use the trained care management HTE model to determine the individuals that benefit the most from enrolling into care management interventions (e.g., result in the greatest reductions in medical costs and/or adverse events by enrolling the individuals into actual care management).

At block 310, the enterprise computing platform 104 provides, for display on a care management computing device, information indicating the plurality of second individuals for the care management interventions. For instance, the enterprise computing platform 104 may provide the information indicating the second individuals (e.g., the name, address, contact information, and/or other information associated with the second individuals) to the care management computing device 108. The care management computing device 108 may display information associated with the second individuals. For example, the care management computing device 108 may be used to enroll the plurality of second individuals into the care management interventions (e.g., actual care management). For instance, the care management computing device 108 may automatically and/or manually enroll the second individuals into the care management interventions and/or perform the care management interventions for the second individuals (e.g., enroll the second individuals into care management interventions, target the second individuals for enrolling into care management interventions, and/or performing actual care management for the second individuals).

In some instances, the care management computing device 108 may display information such as a name and contact information for the second individuals. Afterwards, an operator, nurse, employee, physician, and/or other personnel associated with the enterprise organization may contact the individual. For instance, the operator may call, e-mail, text, and/or otherwise contact the individual to enroll them into care management interventions. Additionally, and/or alternatively, the care management computing device 108 may perform and/or implement care management interventions. For example, after displaying the information for an individual, a nurse may contact that individual to review medications the individual is currently taking and/or provide other types of care management interventions.

FIG. 4 is a simplified block diagram 400 for training the care management HTE model in accordance with one or more examples of the present application. For example, FIG. 4 describes blocks 304 and 306 of process 300 in more detail. FIG. 4 shows a process 400 that may be used by the enterprise computing platform 104 to train the care management HTE models.

In operation, at block 402, the enterprise computing platform 104 obtains covariates for the care management HTE models. For example, as mentioned above, the enterprise computing platform 104 may receive population data for the first individuals from data sources 102. The enterprise computing platform 104 may determine covariates (e.g., covariates information) based on the population data for the first individuals.

At block 404, the enterprise computing platform 104 obtains impact factor datasets for the care management HTE model. For example, based on the population data for the first individuals, the enterprise computing platform 104 may determine impact factor datasets. The impact factor datasets may include and/or indicate impact factor metrics such as chronic condition counts and/or information, medical adherence indicators, and/or other impact factors described above.

At block 406, the enterprise computing platform 104 obtains past population engagement of care management interventions. For instance, based on the population data for the first individuals, the enterprise computing platform 104 may determine past population engagement indicating whether the individuals have been previously enrolled into care management interventions (e.g., actual care management).

At block 407, the enterprise computing platform 104 obtains past population outcomes for care management interventions. For instance, based on the population data for the first individuals, the enterprise computing platform 104 may determine past population outcomes such as clinical outcomes, costs, and/or utilization of the first individuals before and after engaging in care management interventions. For instance, the past population outcomes may be and/or indicate observed outcomes (e.g., cost and/or utilization) after care management interventions. The enterprise computing platform 104 may use the past population outcomes to train the one or more care management ML-AI models.

At block 408, the enterprise computing platform 104 trains one or more care management ML-AI models. For instance, using the covariates, impact factor datasets, past population care management engagement, and/or past population outcomes for care management interventions (e.g., post-engagement financial and/or clinical healthcare outcomes), the enterprise computing platform 104 trains one or more care management ML-AI models.

At block 410, the enterprise computing platform 104 obtains an HTE outcome dataset for the care management HTE model. For instance, the HTE outcome dataset may indicate a performance of the care management HTE model and/or treatment effect model parameter values. For instance, the enterprise computing platform 104 may compare the HTE outcome dataset for the care management HTE model with the past population outcomes for care management interventions to evaluate the performance of the care management HTE model.

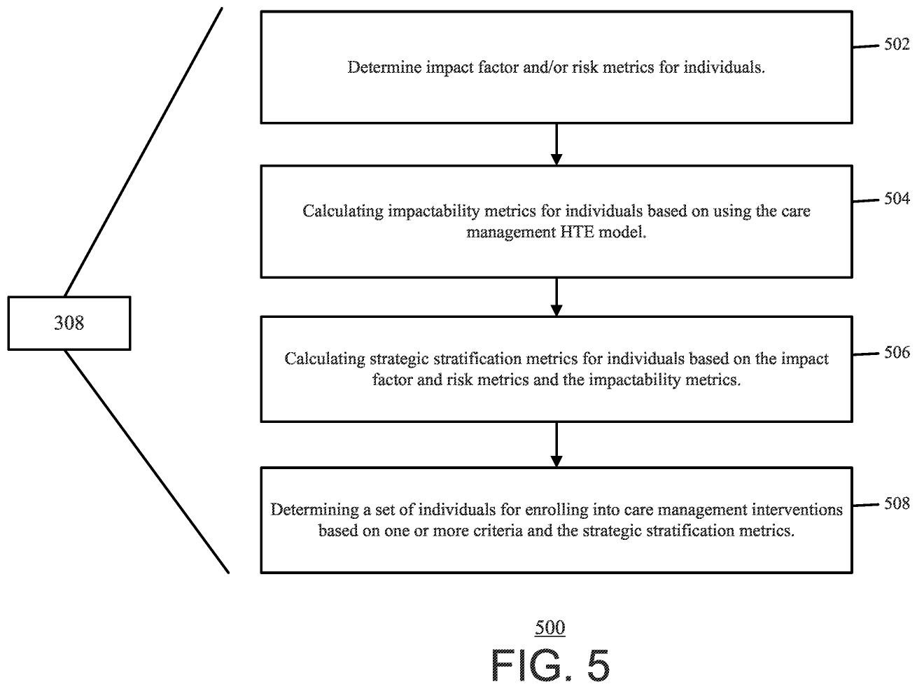

FIG. 5 is another exemplary process for using the care management HTE model for care management interventions in accordance with one or more examples of the present application. In particular, FIG. 5 describes block 308 of process 300 in more detail. For instance, FIG. 5 shows a process 500 that may be used by the enterprise computing platform 104 to determine the plurality of second individuals for enrolling into care management interventions. The process 500 may be performed by the enterprise computing platform 104 shown in FIG. 1. However, it will be recognized that any of the following blocks may be performed in any suitable order and that the process may be performed in any suitable environment. The descriptions, illustrations, and processes of FIG. 5 are merely exemplary and the process may use other descriptions, illustrations, and processes.

At block 502, the enterprise computing platform 104 determines impact factor and/or risk metrics for the individuals (e.g., the third individuals). For instance, the enterprise computing platform 104 may determine additional metrics (e.g., risk metrics such as inpatient risk scores and/or financial risk scores) for the third individuals. For example, the enterprise computing platform 104 may use one or more ML-AI models and/or other algorithms, processes, and/or methods to determine the inpatient risk scores, ER risk scores, and/or financial risk scores. Inpatient, ER and other risk scores may be derived from separately trained models developed to determine generalized risk for specific healthcare outcomes for individuals (e.g. inpatient admission, ER visits, and/or medical costs) without consideration of their enrollment in a care management interventions. In other instances, the enterprise computing platform 104 may receive the additional metrics from another computing platform or system.

At block 504, the enterprise computing platform 104 calculates impactability metrics for individuals based on using the care management HTE model. For example, the enterprise computing platform 104 may input the information associated with the third individuals into the trained care management HTE model (e.g., the care management ML-AI models) to generate output information. The output information may indicate the impactability metrics. For example, the impactability metrics may indicate a risk score and/or a risk value (e.g., a monetary value such as $12).

At block 506, the enterprise computing platform 104 calculates strategic stratification metrics for the individuals based on the impact factor and risk metrics and the impactability metrics. For instance, the impact factor and risk metrics may indicate an inpatient and/or financial risk score (e.g., a risk value such as 97). The impactability metrics may indicate another risk score or risk value (e.g., 12). The enterprise computing platform 104 may determine a strategic stratification metric based on the risk scores such as by combining the risk scores. For instance, the enterprise computing platform 104 may add the inpatient and/or financial risk score (e.g., 97) with the impactability metric (e.g., 12) to determine the strategic stratification metric (e.g., 109). The enterprise computing platform 104 may determine a plurality of strategic stratification metrics for the individuals (e.g., a strategic stratification metric for each individual from the third individuals).

At block 508, the enterprise computing platform 104 determines a set of individuals for enrolling into care management interventions based on one or more criteria and the strategic stratification metrics. For example, the enterprise computing platform 104 may use one or more criteria such as one or more thresholds to determine the set of individuals for enrolling into the care management interventions (e.g., the second individuals). For instance, the enterprise computing platform 104 may enroll individuals based on the strategic stratification metric reaching a certain threshold (e.g., score that is above or below 100). Additionally, and/or alternatively, the one or more criteria may indicate a percentage of individuals from the third individuals to enroll into care management interventions. For instance, the criteria may indicate that 8% of the third individuals are to be enrolled into care management interventions. The enterprise computing platform 104 may use the strategic stratification metrics of the third individuals to determine the top 8% or the bottom 8% of individuals indicated by the strategic stratification metrics. The enterprise computing platform 104 may determine these individuals as the set of individuals for enrolling into the care management interventions.

In some examples, as described above, the enterprise computing platform 104 may use the care management HTE model to identify individuals for care management interventions (e.g., to enroll or engage in care management interventions). For example, the individuals may be associated with MEDICARE, MEDICAID, and/or commercial insurance plans. The enterprise computing platform 104 may train care management HTE models for MEDICARE, MEDICAID, and/or commercial insurance plans. Then, the enterprise computing platform 104 may use the care management HTE models to identify individuals that use MEDICARE, MEDICAID, and/or commercial insurance plans for enrollment into care management interventions. For instance, after training a care management HTE model for MEDICARE (e.g., training the model based on population data from individuals that use MEDICARE), the enterprise computing platform 104 may receive information associated with a group of individuals (e.g., third individuals) that use MEDICARE. The enterprise computing platform 104 may use the care management HTE model for MEDICARE to determine or identify a set of individuals (e.g., second individuals) from the group of individuals. After, the enterprise computing platform 104 may enroll the set of individuals in care management interventions (e.g., provide information to the care management device 108 to enroll the individuals in care management interventions).

Additionally, and/or alternatively, the enterprise computing platform 104 may train one or more care management HTE models for MEDICAID. In some instances, each U.S. state may have different guidelines for MEDICAID. In some examples, the enterprise computing platform 104 may train and use a single care management HTE model for MEDICAID. In other examples, the enterprise computing platform 104 may train and use multiple care management HTE models for MEDICAID (e.g., a first model for one or more U.S. states and a second model for one or more other U.S. states). In some variations, the enterprise computing platform 104 may use different training data for the care management HTE models for the different U.S. states. For instance, the enterprise computing platform 104 may filter the population data for the first individuals based on a geographical location such as the U.S. state that the individual resides in. Based on the filtering, the enterprise computing platform 104 may train the care management HTE models for the different U.S. states. For instance, a first care management HTE model may be trained using training data for one or more U.S. states (e.g., Texas) and a second care management HTE model may be trained using training data for one or more other U.S. states (e.g., California). Subsequently, at blocks 308 and 310, the enterprise computing platform 104 may select the care management HTE model to use based on the residence of the third individuals, and determine the second individuals based on the selected care management HTE model.

A number of implementations have been described. Nevertheless, it will be understood that additional modifications may be made without departing from the scope of the inventive concepts described herein, and, accordingly, other examples are within the scope of the following claims. For example, it will be appreciated that the examples of the application described herein are merely exemplary. Variations of these examples may become apparent to those of ordinary skill in the art upon reading the foregoing description. The inventor expects skilled artisans to employ such variations as appropriate, and the inventor intends for the application to be practiced otherwise than as specifically described herein. Accordingly, this application includes all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

It will further be appreciated by those of skill in the art that the execution of the various machine-implemented processes and steps described herein may occur via the computerized execution of processor-executable instructions stored on a non-transitory computer-readable medium, e.g., random access memory (RAM), read-only memory (ROM), programmable read-only memory (PROM), volatile, nonvolatile, or other electronic memory mechanism. Thus, for example, the operations described herein as being performed by computing devices and/or components thereof may be carried out by according to processor-executable instructions and/or installed applications corresponding to software, firmware, and/or computer hardware.

The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the application and does not pose a limitation on the scope of the application unless otherwise claimed. No language in the specification should be construed as indicating any non-claimed element as essential to the practice of the application.

1. A method, comprising:
receiving, by a computing platform and from a plurality of data sources, population data for a plurality of first individuals;
standardizing, by the computing platform, the population data to determine training data for a care management heterogeneous treatment effect (HTE) model, wherein the training data comprises a plurality of impact factor datasets for the plurality of first individuals;
training, by the computing platform, the care management HTE model using the training data, wherein the care management HTE model comprises one or more care management machine learning-artificial intelligence (ML-AI) models;
determining, by the computing platform, a plurality of second individuals for care management interventions based on using the trained care management HTE model; and
providing, by the computing platform and for display on a care management computing device, information indicating the plurality of second individuals for the care management interventions.

2. The method of claim 1, wherein standardizing the population data comprises:
determining a plurality of covariates for the care management HTE model based on the population data;
determining the plurality of impact factor datasets for the care management HTE model based on the population data; and
determining past population engagement in the care management interventions for the plurality of first individuals,
wherein training the care management HTE model is based on the plurality of covariates, the plurality of impact factor datasets, and the past population engagement.

3. The method of claim 2, wherein standardizing the population data further comprises:
determining an HTE outcome dataset for the care management HTE model based on training the care management HTE model using the plurality of covariates, the plurality of impact factor datasets, and the past population engagement, wherein the HTE outcome dataset comprises associated treatment effect model parameter values.

4. The method of claim 2, wherein determining the plurality of impact factor datasets comprises determining a plurality of impact factor metrics, wherein the plurality of impact factor metrics comprise fall risks, emergency room (ER) risks, medical adherence indicators, chronic condition counts, mental illness indicators, usage of durable medical equipment (DME), new onset of diseases, and/or drug safety indicators.

5. The method of claim 2, wherein training the care management HTE model using the training data comprises:
using the plurality of impact factor datasets and the plurality of covariates to train the one or more care management ML-AI models, wherein the plurality of impact factor datasets and the plurality of covariates are features for the one or more care management ML-AI models.

6. The method of claim 5, wherein standardizing the population data further comprises:
determining past population outcomes for the care management interventions for the plurality of first individuals, wherein the past population outcomes indicate post-engagement clinical and/or financial healthcare outcomes for the plurality of first individuals after undergoing the care management interventions, and
wherein using the plurality of impact factor datasets and the plurality of covariates to train the one or more care management ML-AI models further comprises using the plurality of impact factor datasets, the plurality of covariates, the past population engagement, and the past population outcomes to train the one or more care management ML-AI models.

7. The method of claim 1, wherein determining the plurality of second individuals for the care management interventions based on using the trained care management HTE model comprises:
determining a plurality of new impact factors and a plurality of new covariate datasets associated with the plurality of second individuals;
inputting the plurality of new impact factors and the plurality of new covariate datasets into the one or more care management ML-AI models to determine output information for the plurality of second individuals; and
determining the plurality of second individuals for the care management interventions based on the output information.

8. The method of claim 7, wherein determining the plurality of second individuals for the care management interventions comprises:
combining the output information with additional metrics to generate combined strategic stratification metrics associated with the plurality of second individuals; and
determining the plurality of second individuals for enrolling into the care management interventions based on comparing the combined strategic stratification metrics with one or more strategic stratification threshold values.

9. The method of claim 1, wherein the plurality of first individuals and the plurality of second individuals are enrolled into MEDICARE.

10. The method of claim 1, wherein the plurality of first individuals and the plurality of second individuals are enrolled into MEDICAID.

11. The method of claim 1, wherein the plurality of first individuals and the plurality of second individuals are enrolled into a commercial plan.

12. An enterprise computing platform, comprising:
one or more processors; and
a non-transitory computer-readable medium having processor-executable instructions stored thereon, wherein the processor-executable instructions, when executed by the one or more processors, facilitate:
receiving, from a plurality of data sources, population data for a plurality of first individuals;
standardizing the population data to determine training data for a care management heterogeneous treatment effect (HTE) model, wherein the training data comprises a plurality of impact factor datasets for the plurality of first individuals;
training the care management HTE model using the training data, wherein the care management HTE model comprises one or more care management machine learning-artificial intelligence (ML-AI) models;
determining a plurality of second individuals for care management interventions based on using the trained care management HTE model; and
providing, for display on a care management computing device, information indicating the plurality of second individuals for the care management interventions.

13. The enterprise computing platform of claim 12, wherein standardizing the population data comprises:
determining a plurality of covariates for the care management HTE model based on the population data;
determining the plurality of impact factor datasets for the care management HTE model based on the population data; and
determining past population engagement in the care management interventions for the plurality of first individuals,
wherein training the care management HTE model is based on the plurality of covariates, the plurality of impact factor datasets, and the past population engagement.

14. The enterprise computing platform of claim 13, wherein standardizing the population data further comprises:
determining an HTE outcome dataset for the care management HTE model based on training the care management HTE model using the plurality of covariates, the plurality of impact factor datasets, and the past population engagement, wherein the HTE outcome dataset comprises associated treatment effect model parameter values.

15. The enterprise computing platform of claim 13, wherein determining the plurality of impact factor datasets comprises determining a plurality of impact factor metrics, wherein the plurality of impact factor metrics comprise fall risks, emergency room (ER) risks, medical adherence indicators, chronic condition counts, mental illness indicators, usage of durable medical equipment (DME), new onset of diseases, and/or drug safety indicators.

16. The enterprise computing platform of claim 13, wherein training the care management HTE model using the training data comprises:
using the plurality of impact factor datasets and the plurality of covariates to train the one or more care management ML-AI models, wherein the plurality of impact factor datasets and the plurality of covariates are features for the one or more care management ML-AI models.

17. The enterprise computing platform of claim 16, wherein standardizing the population data further comprises:
determining past population outcomes for the care management interventions for the plurality of first individuals, wherein the past population outcomes indicate post-engagement clinical and/or financial healthcare outcomes for the plurality of first individuals after undergoing the care management interventions, and
wherein using the plurality of impact factor datasets and the plurality of covariates to train the one or more care management ML-AI models further comprises using the plurality of impact factor datasets, the plurality of covariates, the past population engagement, and the past population outcomes to train the one or more care management ML-AI models.

18. The enterprise computing platform of claim 12, wherein determining the plurality of second individuals for the care management interventions based on using the trained care management HTE model comprises:
determining a plurality of new impact factors and a plurality of new covariate datasets associated with the plurality of second individuals;
inputting the plurality of new impact factors and the plurality of new covariate datasets into the one or more care management ML-AI models to determine output information for the plurality of second individuals; and
determining the plurality of second individuals for the care management interventions based on the output information.

19. The enterprise computing platform of claim 18, wherein determining the plurality of second individuals for the care management interventions comprises:
combining the output information with additional metrics to generate combined strategic stratification metrics associated with the plurality of second individuals; and
determining the plurality of second individuals for enrolling into the care management interventions based on comparing the combined strategic stratification metrics with one or more strategic stratification threshold values.

20. A non-transitory computer-readable medium having processor-executable instructions stored thereon, wherein the processor-executable instructions, when executed, facilitate:
receiving, from a plurality of data sources, population data for a plurality of first individuals;
standardizing the population data to determine training data for a care management heterogeneous treatment effect (HTE) model, wherein the training data comprises a plurality of impact factor datasets for the plurality of first individuals;
training the care management HTE model using the training data, wherein the care management HTE model comprises one or more care management machine learning-artificial intelligence (ML-AI) models;
determining a plurality of second individuals for care management interventions based on using the trained care management HTE model; and
providing, for display on a care management computing device, information indicating the plurality of second individuals for the care management interventions.
